(19) United States
(12) Patent Application Publication
(10) Pub. No.: US 2009/0205661 A1
(43) Pub. Date: Aug. 20, 2009

(54) SYSTEMS AND METHODS FOR EXTENDED VOLUME RANGE VENTILATION

(75) Inventors: Robert Stephenson, Carlsbad, CA (US); Jon Guy, Carlsbad, CA (US); Gabriel Sanchez, Valley Center, CA (US); David P. Winter, Encinitas, CA (US)

Correspondence Address: NELLCOR PURITAN BENNETT LLC;ATTN: IP LEGAL, 60 MIDDLETOWN AVENUE, NORTH HAVEN, CT, 06473, US

(73) Assignee: Nellcor Puritan Bennett LLC, Boulder, CO (US)

(21) Appl. No.: 12/371,877

(22) Filed: Feb. 16, 2009

Related U.S. Application Data
(60) Provisional application No. 61/030,103, filed on Feb. 20, 2008.

(51) Int. Cl. A61M 16/12; A61M 16/20

(52) U.S. Cl. 128/204.21; 128/204.18; 128/205.24

(57) ABSTRACT

Various embodiments of the present invention provide systems, methods and devices for delivering a defined gas mixture to a recipient. For example, various embodiments of the present invention provide ventilators that include at least two gas sources, a gas outlet and a differential flow transfer element. The differential flow transfer element receives one component gas from one of the gas sources at a first flow rate, and another component gas from the other gas source at a second flow rate. The differential flow transfer element distributes a mixture that includes at least the aforementioned component gases at a third flow rate via the gas outlet. The third flow rate is less than the sum of the first flow rate and the second flow rate.

### RELATED APPLICATION

This application claims priority from U.S. Patent Application No.61/030,103 which was filed on Feb. 20, 2008, and is incorporated herein by reference in its entirety.

### BACKGROUND OF THE INVENTION

The present invention is related to ventilators, and more particularly to systems and methods for mixing gases in a ventilator.

Modern ventilators are designed to ventilate a patient's lungs with gas, and to thereby assist the patient when the patient's ability to breathe on their own is somehow impaired. In a simple situation, a ventilator receives a defined gas mixture at a constant rate, and provides the defined gas mixture to the patient at the same constant rate. Such a process aids a patient in their inspiratory efforts; however, it requires pre-mixed gases that can be expensive, inflexible and inconvenient.

More sophisticated ventilators provide for mixing gases from different gas sources to yield a desired gas mixture for a patient. In particular, the introduction of each of the gases is controlled by a respective flow delivery valve. The flow delivery valves are configured in parallel, with the outputs of each of the flow delivery valves provided to a common output. Thus, the total flow of gas to the patient is equal to the sum of all gases passing through the flow delivery valves, and the content of the gas provided to the patient is governed by the relative flow of each of the flow delivery valves. In such ventilators, the accuracy of the gas content and volume provided to a patient is limited by the accuracy of each of the flow delivery valves. Therefore, these ventilators work reasonably well where the flow of each of the constituent gases is well within the metering ability of the flow delivery valves. For example, a gas mixture comprising air with a forty percent oxygen content to be delivered to an adult patient may be accurately delivered as both the air and the oxygen are incorporated at substantial flows. In contrast, the accuracy of a gas mixture comprising air with a twenty-two percent oxygen content to be delivered to a neonatal patient may be poor due to the insubstantial amount of oxygen combined with the air.

Hence, there exists a need in the art for advanced ventilation systems, and methods for using such.

### BRIEF SUMMARY OF THE INVENTION

The present invention is related to ventilators, and more particularly to systems and methods for mixing gases in a ventilator.

Various embodiments of the present invention provide ventilators that include at least two gas sources, a gas outlet and a differential flow transfer element. The differential flow transfer element receives one component gas from one of the gas sources at a first flow rate, and another component gas from the other gas source at a second flow rate. The differential flow transfer element distributes a mixture that includes at least the aforementioned component gases at a third flow rate via the gas outlet. In various instances, the differential flow transfer element may be an accumulator. In some such cases, the accumulator may be designed to operate at a pressure of between five and fifteen psi. In particular cases, the accumulator may be designed to operate between nine and twelve psi.

In the aforementioned embodiment, the third flow rate is less than the sum of the first flow rate and the second flow rate. In various instances of the aforementioned embodiments, the sum of the volume of the first component gas received from the first gas source and the volume of the second component gas received from the second gas source approximately equals the volume of the mixture provided via the gas outlet when measured over a period extending two or more consecutive inlet periods. In some cases, the first flow rate and the second flow rate are different. In one or more instances of the aforementioned embodiments the third flow rate exhibits a flow and periodicity consistent with a human breathing pattern. In such instances, one or both of the first flow rate and the second flow rate is substantially higher than the third flow rate, but with a longer period.

In various instances of the aforementioned embodiments, the differential flow transfer element receives the first component gas from the first gas source via a flow delivery module including a flow delivery valve, and is programmable to deliver the first component gas at the first flow rate. In some cases, the flow delivery module further includes a flow sensor that is operable to sense the flow of the first component gas into the differential flow transfer element. The first component gas may be, but is not limited to, air, oxygen, heliox, or helium.

Other embodiments of the present invention provide gas delivery systems that include a differential flow transfer element, a processor and a computer readable medium including instructions executable by the processor. The differential flow transfer element is coupled to a first component gas via a first flow valve, to a second component gas via a second flow valve, and to an outlet via a third flow valve. The instructions are executable by the processor to operate the first flow valve intermittently at a first flow rate and the second flow valve intermittently at a second flow rate. Such operation yields a defined mixture including the first component gas and the second component gas in the differential flow transfer element. In addition, the instructions are executable to operate the third flow valve intermittently at a third flow rate to deliver the defined mixture including the first component gas and the second component gas from the differential flow transfer element to the outlet. The third flow rate is less than the sum of the first flow rate and the second flow rate.

In various instances of the aforementioned embodiments, the computer readable medium further includes instructions executable by the processor to receive an indication of the volume of the first component gas traversing the first flow valve; receive an indication of the volume of the second component gas traversing the second flow valve; receive an indication of the volume of the defined mixture traversing the third flow valve; and based thereon to calculate an amount of at least one constituent gas in the differential flow transfer element.

In some instances of the aforementioned embodiment, the computer readable medium further includes instructions executable by the processor to receive a request for the defined mixture, and to calculate the first flow rate and the second flow rate. In some cases, the instructions are further executable to receive a request for another defined mixture including the first component gas and the second component gas, and to operate the first and second flow valves intermittently to yield the updated defined mixture in the differential flow transfer element. In some such cases, a dump valve is opened to allow the preceding defined mixture in the differential flow transfer element to exhaust. In other cases, the preceding defined mixture is modified until it becomes the updated defined mixture. In such cases, the computer readable medium may further include instructions executable by the processor to receive an indication of the pressure in the differential flow transfer element, and to calculate an amount of at least one constituent gas in the differential flow transfer element based at least in part on the pressure in the differential flow transfer element.

Yet other embodiments of the present invention include methods for providing breathable gas to a recipient. An accumulator is provided that is coupled to a first component gas via a first flow valve, to a second component gas via a second flow valve, and to an outlet via a third flow valve. The methods include receiving a request for a defined mixture including the first component gas and the second component gas, operating the first flow valve intermittently at a first flow rate and the second flow valve intermittently at a second flow rate to yield the defined mixture in the accumulator, and operating the third flow valve intermittently at a third flow rate to deliver the defined mixture from the accumulator to the outlet. The third flow rate is less than the sum of the first flow rate and the second flow rate, and over a period extending two or more inlet periods, the sum of the volume of the first component gas received via the first flow valve the volume of the second component gas received via the second flow valve approximately equals the volume of the defined mixture provided via the third flow valve.

This summary provides only a general outline of some embodiments of the invention. Many other objects, features, advantages and other embodiments of the invention will become more fully apparent from the following detailed description, the appended claims and the accompanying drawings.

### BRIEF DESCRIPTION OF THE DRAWINGS

A further understanding of the various embodiments of the present invention may be realized by reference to the figures which are described in remaining portions of the specification. In the figures, like reference numerals may be used throughout several of the figures to refer to similar components. In some instances, a sub-label consisting of a lower case letter is associated with a reference numeral to denote one of multiple similar components. When reference is made to a reference numeral without specification to an existing sub-label, it is intended to refer to all such multiple similar components.

FIG. 1 is a block diagram of a ventilation system in accordance with various embodiments of the present invention;

FIGS. 2 depicts a ventilator feedback and control system in accordance with one or more embodiments of the present invention;

FIGS. 3a-3c are flow diagrams depicting operation of a ventilation system in accordance with some embodiments of the present invention; and

FIG. 4 is a timing diagram graphically depicting an example of intermittent volume of component gas flows into a differential flow transfer element, and an intermittent volume of mixed gas flow from the differential flow transfer element that may be achieved in accordance with one or more embodiments of the present invention.

### DETAILED DESCRIPTION OF THE INVENTION

The present invention is related to ventilators, and more particularly to systems and methods for mixing gases in a ventilator.

Various embodiments of the present invention provide ventilators that are capable of receiving one or more component gases at programmed flow rates to yield a desired gas mixture, and for distributing the gas mixture at an output flow rate. The input flow rate is the sum of the flow rates for the component gases introduced to the ventilator, and is not necessarily the same as the output flow rate. Particular embodiments of the present invention exhibit an output flow rate that is substantially less than the combined input flow rate for a given time period. Thus, as one example, the input flow rate may be sustained for thirty seconds and then paused for three minutes at the same time that the output flow rate is consistently producing the gas mixture to a recipient at a flow and periodicity consistent with human breathing patterns. In various instances of the aforementioned embodiments, a differential flow transfer element is used to accommodate a substantial difference between the input and output flow rates while conserving the received input gases. In such instances, reception of the input gases and production of the output gas may be intermittent, with the off period of the inlet gases being substantially greater than the off period of the outlet gas.

As used herein, the phrase “constituent gas” is used in its broadest sense to mean any elemental gas that is included in a gas mixture. Thus, a constituent gas may include, but is not limited to, oxygen, nitrogen and helium. Based on the disclosure provided herein, one of ordinary skill in the art will recognize a variety of different constituent gases that may be used in relation to different embodiments of the present invention. Further, as used herein, the phrase “component gas” is used in its broadest sense to mean any gas that is provided via an inlet of a ventilator. Thus, a component gas may be, but is not limited to, air, heliox, helium or oxygen Based on the disclosure provided herein, one of ordinary skill in the art will recognize a variety of different component gases that may be used in relation to different embodiments of the present invention. It should be noted that a component gas may comprise a number of constituent gases. For example, air may be a component gas that includes, among other things, constituent gases of nitrogen and oxygen. Some embodiments of the present invention utilize a gas profile associated with each component gas that indicates the various constituent gases by volume Thus, for example, a gas profile associated with air may indicate that air includes the following constituent gases by volume: nitrogen (78%), oxygen (20.95%), and argon (0.93%). As another example, a gas profile associated with heliox may indicate that a particular type of heliox includes the following constituent gases by volume: helium (x %) and oxygen (y %). In one particular case, heliox may include 80% helium and 20% oxygen. Based on the disclosure provided herein, one of ordinary skill in the art will recognize a variety of gas profiles that may be used depending upon which component gases are selected for use in relation to ventilators in accordance with the various embodiments of the present invention.

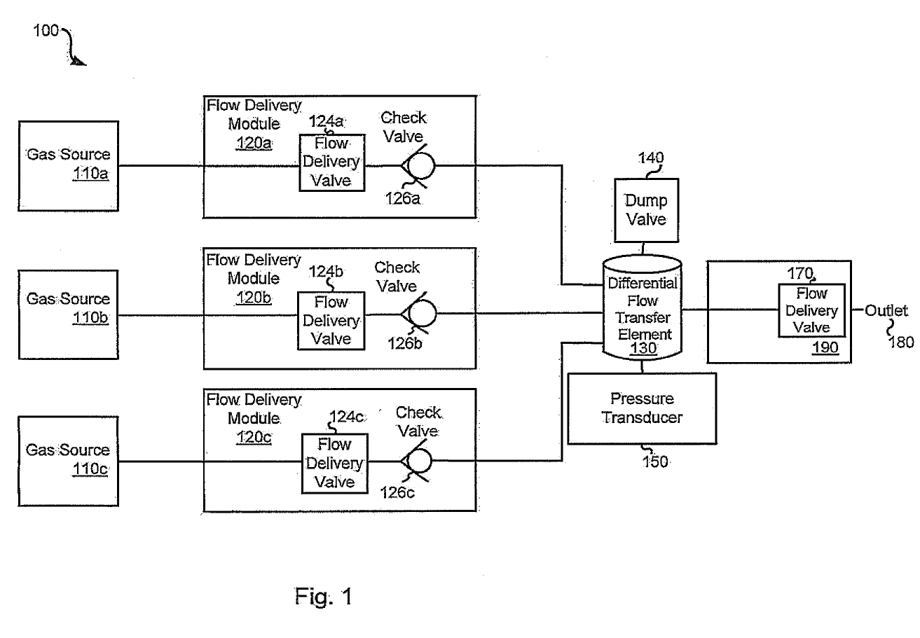

Turning to FIG. 1, a block diagram of a ventilation system 100 is depicted in accordance with various embodiments of the present invention. Ventilation system 100 includes a differential flow transfer element 130 that receives component gases from one or more of gas sources 110, and provides a mixture of the component gases to an outlet 180. As used herein, the phrase “gas source” is used in its broadest sense to mean any inlet through which an associated gas may be introduced to ventilation system 100. The resulting gas mixture includes a prescribed level of one or more constituent gases derived from the inlet component gases. In some particular embodiments of the present invention, differential flow transfer element 130 is an accumulator that operates between five and fifteen psi. In one particular embodiment of the present invention, differential flow transfer element 130 is an accumulator that operates between nine and twelve psi. Based on the disclosure provided herein, one of ordinary skill in the art will recognize a variety of flow transfer elements and/or particular accumulators that may be utilized in relation to different embodiments of the present invention. It should be noted that while ventilation system 100 is shown having three distinct gas sources 110, that different embodiments of the present invention may allow for receiving gases from more or fewer than three gas sources. Gas sources 110 may include, but are not limited to, a helium source, an oxygen source, an air source, and/or a heliox source.

A component gas from gas source 110a is introduced to differential flow transfer element 130 via a flow delivery module 120a; another component gas from gas source 110b is introduced to differential flow transfer element 130 via a flow delivery module 120b; and yet another component gas from gas source 110c is introduced to differential flow transfer element 130 via a flow delivery module 120c. Each flow delivery module 120 includes a flow delivery valve 124 and a check valve 126. Flow delivery valve 124 may be any valve capable of governing the flow of gas passed from the associated gas source 110 to differential flow transfer element 130. In some instances, one or more of flow delivery valves 124 may be programmable. In some particular embodiments of the present invention, flow delivery valve 124 is a proportional solenoid type valve capable of delivering from 0 to 125 L/min. Check valve 126 may be any valve that is capable of allowing gas to flow in one direction, but not another. In this case, check valves 126 preclude gas from flowing from differential flow transfer element 130 to any of gas sources 110. Based on the disclosure provided herein, one of ordinary skill in the art will recognize a variety of particular valve types and flow sensors that may be utilized in relation to embodiments of the present invention. In various cases, flow delivery modules 120 may further include a flow sensor (not shown) that may be any sensor known in the art, such as a differential pressure flow sensor, that is capable of determining the flow of gas passing through or by the sensor.

As shown, differential flow transfer element 130 is coupled to a dump valve 140 and a pressure transducer 150. Pressure transducer 150 is operable to determine the pressure build up in differential flow transfer element 130, and may be any of a number of types of pressure transducers that are known in the art. Dump valve 140 is operable to release gas maintained in differential flow transfer element 130 into the atmosphere. Dump valve 140 may be any type of valve known in the art that is capable of releasing gas from differential flow transfer element 130.

Ventilation system 100 also includes an output delivery module 190 that is responsible for providing gas from differential flow transfer element 130 to outlet 180. Output delivery module 190 includes a flow delivery valve 170. Flow delivery valve 170 may be any valve capable of programmably controlling the flow of gas passed from differential flow transfer element 130 to outlet 180. In one particular embodiment of the present invention, flow valve 170 is a proportional solenoid type valve capable of delivering controlled flow from 0 to 200 L/min. Flow sensor 160 may be any sensor known in the art that is capable of determining the flow of gas passing through or by the sensor. In some cases, output delivery module 190 further includes a flow sensor (not shown), such as a differential flow sensor, or other flow sensor known in the art.

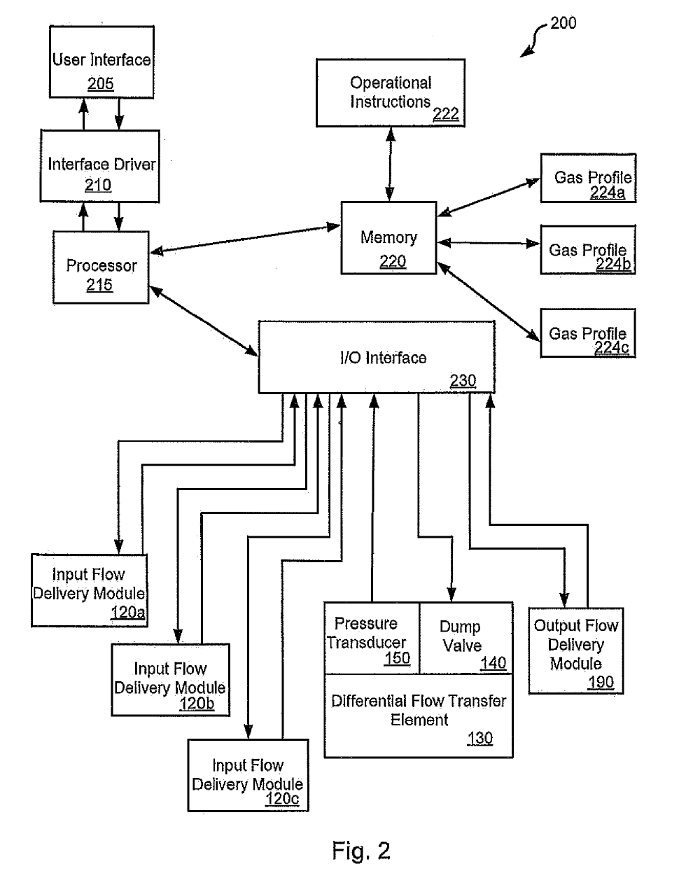

Turning to FIG. 2, a control diagram depicts a ventilator feedback and control system 200 in accordance with one or more embodiments of the present invention that is capable of governing the reception, mixing and distribution of gases. Feedback and control system 200 includes a user interface 205 that is controlled by a processor 215 via an interface driver 210. In some embodiments of the present invention, user interface 205 is a touch screen interface that is capable of receiving user commands that are provided to processor 215, and is capable of providing a user display based on information provided from processor 215. It should be noted that the aforementioned touch screen user interface is merely exemplary, and that one of ordinary skill in the art will recognize a variety of user interface devices or systems that may be utilized in relation to different embodiments of the present invention.

Processor 215 may be any processor known in the art that is capable of receiving feedback from user interface 205, executing various operational instruction 222 maintained in a memory 220, and processing various I/O via an I/O interface 230. I/O interface 230 allows for providing output control to each of input flow delivery modules 120, dump valve 140, and output flow delivery module 190. Further, I/O interface 230 allows for receiving pressure information from pressure transducer 150.

Memory 220 includes operational instructions 222 that may be software instructions, firmware instructions or some combination thereof. Operational instructions 222 are executable by processor 215, and may be used to cause processor 215 to control a ventilator in a programmed manner. In addition, memory 220 includes a number of gas profiles 224 that identify the composition of gases introduced via each of flow delivery modules 120 (e.g., the constituent gas composition of gas sources 110). Thus, for example, where flow delivery module 120a is associated with an oxygen source, flow delivery module 120b is associated with a helium source, and flow delivery module 120c is associated with an air source, gas profile 224a would indicate pure oxygen, gas profile 224b would indicate pure helium, and gas profile 224c would indicate the constituent gases included in air and their respective ratios (e.g., 78% nitrogen, 20.95% oxygen, and 0.93% argon).

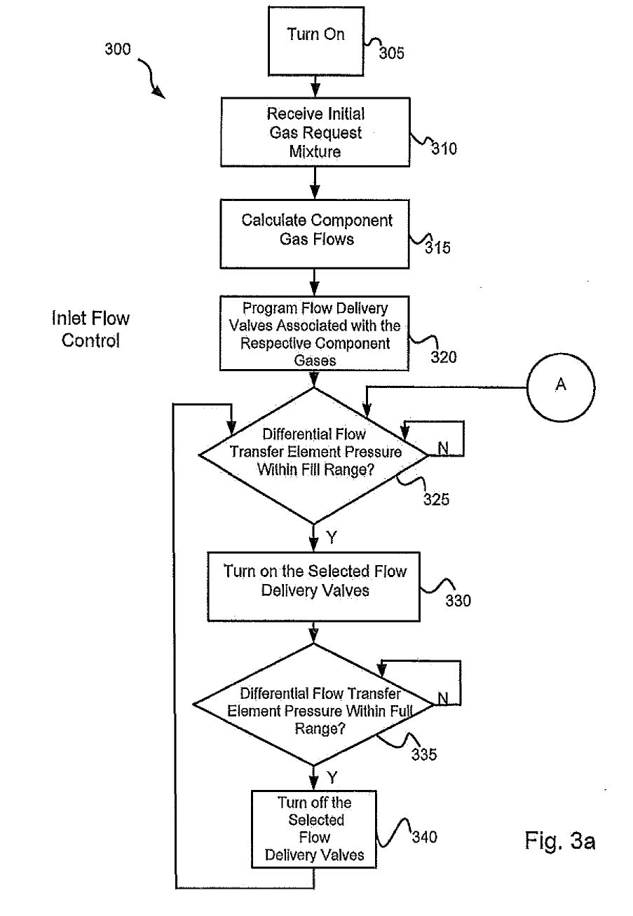

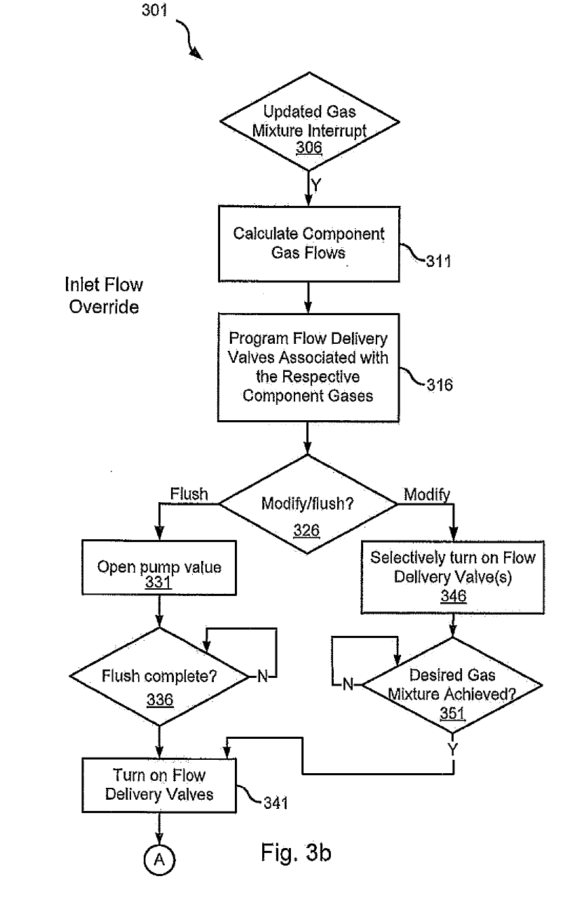

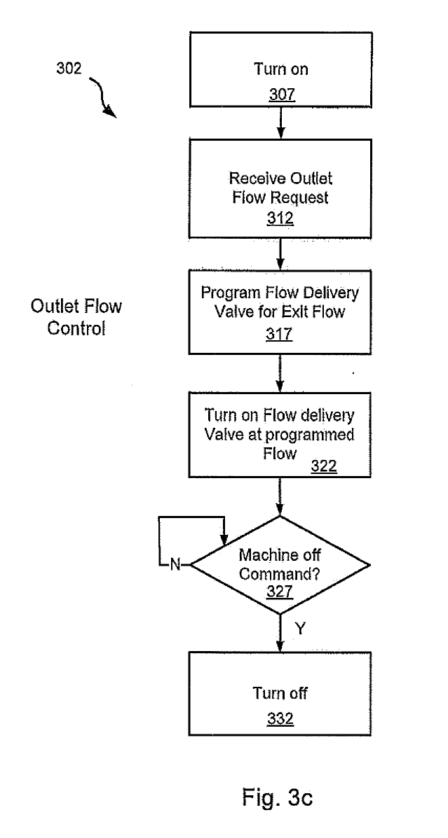

Turning to FIGS. 3a-3c, three flow diagrams 300, 301, 302 depict operation of a ventilation system in accordance with some embodiments of the present invention, Flow diagrams 300, 301, 302 each represent a distinct process. In particular, flow diagram 300 depicts control of the introduction of component gases to differential flow transfer element 130, and flow diagram 302 depicts control of providing the gas mixture from differential flow transfer element 130 to outlet 180. Both of these processes proceed in parallel to the other, and allow for filling differential flow transfer element 130 intermittently at a relatively high rate, and for providing the gas mixture from differential flow transfer element 130 at a lower more constant rate. The input rate and the output rate may be separately selected to satisfy competing concerns. For example, the input rate may be selected to satisfy one or more metering limitations of flow delivery valves 124 and the output rate may be selected to satisfy gas delivery requirements of a recipient. Flow diagram 301 is an interrupt process that overrides the operation of flow diagram 300 whenever a request to change the gas mixture delivered by the ventilator is received. Flow diagrams 300, 301, 302 are described with reference to the systems of FIG. 1 and FIG. 2, however, it should be noted that the operation represented by the flow diagrams may be implemented in relation to different ventilation systems and/or the ventilator control systems.

Following flow diagram 300, ventilator system 100 is powered on (block 305 ). This may be accomplished using any method to power on a ventilator that is known in the art including, but not limited to, applying power via an on/off switch or resetting the machine. Upon power up, a user is queried for a desired output gas mixture. In response, a request for a desired gas mixture is received (block 310). In some cases, this process may include displaying the user query via user interface 205 and receiving the user's response via the same interface. Based on the disclosure provided herein, one of ordinary skill in the art will recognize a variety of querying displays and associated responses that may be used and processed in accordance with various embodiments of the present invention.

The flow of the various component gases required to derive the requested gas mixture is calculated by processor 215 (block 315). In one particular embodiment of the present invention, calculating the respective flows includes selecting a base component gas at a nominal flow, and then selecting one or more component gases and associated flows to be added to the base gas such that the desired gas mixture is yielded in differential flow transfer element 130. In some cases, the base component gas may be chosen to be the available component gas that is most similar to the desired output mixture. This can be done by processor 215 accessing each of gas profiles 224 from memory 220 and comparing the respective gas profiles against the desired output gas. Thus, for example, where the desired gas mixture is air with an increased oxygen percentage by volume, the base component gas may be chosen to be air (i.e., air with an oxygen content of 20.95%) at a particular flow rate. To yield the desired increase in oxygen content, an oxygen component gas may be selected with a flow rate determined by the following equation:

$$Component Oxygen Flow = [ \frac{Desired Oxygen Concentration}{20.95 \%} - 1 ] Component Air Flow ;$$

Thus, for example, where the desired gas mixture is air with a twenty-two percent oxygen concentration by volume, air component gas may be selected to flow at a nominal one liter per minute. To yield a twenty-two percent oxygen concentration, a flow of oxygen component gas at 0.0501 liters per minute is calculated. In some cases, flow delivery valves 124 and/or flow sensors 122 may not be able to accurately deliver or meter such a low gas flow. As the expiratory process (i.e., the process of flow diagram 302) is decoupled from the inspiratory process (i.e., the process of flow diagram 300) by differential flow transfer element 130, it is possible to arbitrarily increase the flow of both the air component gas and the oxygen component gas by the same factor (k) to bring both flows within accurately deliverable ranges Thus, for example, both flows may be multiplied by a factor of k yielding an inlet flow of k liters/minute of air component gas, and 0.0501 k liters/minute of oxygen component gas which are both accurately measurable with a standard allowable error. As will become more apparent after the discussion of flow diagram 301 and flow diagram 302, the aforementioned inlet flows may be used to deliver mixed gas to an adult patient or a neonatal patient as the inlet flow is decoupled from the outlet flow by differential flow transfer element 130.

As another example where the desired gas mixture is heliox with a defined oxygen concentration by volume, the base component gas may be chosen to be helium at a particular flow rate. Again, the base component gas may be chosen as the available component gas defined by a gas profile that is most similar to the desired output mixture. To yield the desired level of oxygen, an oxygen component gas may be selected with a flow rate determined by the following equation:

$$Component Oxygen Flow = \frac{\begin{matrix}[ Desired Oxygen Concentration ] \\ Component Helium Flow\end{matrix}}{\begin{matrix}1 - \\ Desired Oxygen Concentration\end{matrix}} ;$$

Thus, for example, where the desired gas mixture is heliox with a ten percent oxygen concentration by volume, helium component gas may be selected to flow at a nominal one liter per minute. To yield a ten percent oxygen concentration, a flow of oxygen component gas at 0.111 liters per minute is calculated. Again, in some cases, flow delivery valves 124 and/or flow sensors 122 may not be able to accurately deliver such a low gas flow. Both flows may be multiplied by a factor k yielding an inlet flow of k liters/minute of helium component gas, and 0.111 k liters/minute of oxygen component gas which are both accurately measurable with a standard allowable error. Again, the aforementioned inlet flows may be used to deliver mixed gas to an adult patient or a neonatal patient as the inlet flow is decoupled from the outlet flow by differential flow transfer element 130.

As yet another example, the desired gas mixture may be air with a defined oxygen concentration and a defined helium concentration. In such a case the base component gas may be chosen to be air at a nominal flow rate. In addition, both oxygen and helium component gases would be selected to flow to differential flow transfer element 130 at calculated rates to yield the desired gas mixture. It should be noted that the aforementioned examples are merely exemplary, and that one of ordinary skill in the art will recognize a variety of other component gases and mixtures thereof that are possible through use of one or more embodiments of the present invention.

With the desired flow of each component gas calculated (block 315), the respective flow delivery valves are programmed to allow the calculated flow to pass (block 320). Thus, using the example above for air with a twenty-two percent oxygen concentration by volume where air component gas is provided via gas source 110a and oxygen component gas is provided via gas source 110b, flow delivery valve 124a may be programmed to allow k liters/minute of air component gas to pass and flow delivery valve 124b is programmed to allow 0.0501 k liters/minute of oxygen component gas to pass. Flow delivery valve 124c is shut or turned off. This results in a gas mixture of air with the twenty-two percent oxygen concentration by volume flowing into differential flow transfer element 130 at a relatively high fill rate. Using the other example above where the desired gas mixture is heliox with a ten percent oxygen concentration by volume where oxygen component gas is provided via gas source 110b and helium component gas is provided via gas source 110c, flow delivery valve 124c may be programmed to allow k liters/minute of helium component gas to pass and flow delivery valve 124b is programmed to allow 0.111 k liters/minute of oxygen component gas to pass. Flow delivery valve 124a is shut or turned off. This results in a gas mixture of heliox with the ten percent oxygen concentration by volume flowing into differential flow transfer element 130 at a relatively high fill rate. Again, based on the disclosure provided herein, one of ordinary skill in the art will recognize a variety of other gas mixtures that may be flowed to differential flow transfer element 130 using embodiments of the present invention. Depending upon the desired gas mixture, component gases from a single gas source, from two different gas sources, or from thee or more gas sources may be flowed into differential flow transfer element 130.

It is determined whether the pressure in differential flow transfer element 130 is within a fill range (block 325). The pressure in flow transfer element is ascertained by reading pressure transducer 150. Where the pressure in differential flow transfer element 130 is outside of the fill range (block 325), the process of filling remains paused. Alternatively, where the pressure in differential flow transfer element 130 is within the fill range (block 325), the flow delivery valves associated with component gases selected for inclusion in the desired gas mixture are turned on to allow the gas flow calculated and programmed in blocks 315, 320 above (block 330). Once the selected flow delivery valves 124 are turned on to allow filling of differential flow transfer element 130 (block 330), it is determined whether the pressure within a full range (block 335). Where the pressure within differential flow transfer element 130 is outside of the full range (block 335), the process of filling continues. Alternatively, where the pressure within differential flow transfer element 130 is within the full range (block 335), the flow delivery valves are turned off to pause the filling process (block 340). The fill process remains paused until the pressure again comes within the fill range (block 325).

As an example, in an embodiment of the present invention where differential flow transfer element 130 is an accumulator that operates between a lower pressure and an upper pressure, the fill range may be defined as the range between the lower pressure and the upper pressure. The lower pressure is referred to herein as a “turn-on” pressure, and the upper pressure is referred to herein as a “turn-off” pressure. Determining whether the differential flow transfer element 130 is within a fill range may include determining whether the pressure in the accumulator is below the turn-on pressure, and determining whether the differential flow transfer element 130 is within a full range may include determining whether the pressure in the accumulator is at or above the upper pressure. In such a case, the accumulator would be filled (block 330) until the turn-off pressure is achieved (block 335) at which time the fill process would be paused (block 340). Once the pressure in the accumulator drops below the turn-on pressure (block 325), the process of filling would be restarted (block 330) and continue until the turn-off pressure is achieved (block 335). Based on the disclosure provided herein, one of ordinary skill in the art will recognize a variety of turn-on and turn-off pressures that may be utilized depending upon the particular accumulator used to implement differential flow transfer element 130.

Again, flow diagram 301 is an interrupt process that overrides the operation of flow diagram 300 whenever a request to change the gas mixture delivered by the ventilator is received. Following flow diagram 301, it is determined whether an updated gas mixture interrupt has been received (block 306). Such an interrupt may be received, for example, whenever a user enters a modification to an earlier gas mixture request via user interface 205. The interrupt may be received using any interrupt scheme known in the art including, but not limited to, using a polling scheme where processor 215 periodically reviews an interrupt register, or using an asynchronous interrupt port of processor 215. Based on the disclosure provided herein, one of ordinary skill in the all will recognize a variety of interrupt schemes that may be used in relation to different embodiments of the present invention. Where an updated gas mixture interrupt is received (block 306), the process of flow diagram 300 is interrupted. During the interruption, the flow of the various component gases required to derive the requested gas mixture is calculated by processor 215 (block 311). As with that described in relation to flow diagram 300, this process may include selecting a base component gas at a nominal flow, and then selecting one or more component gases and associated flows to be added to the base gas such that the desired gas mixture is yielded in differential flow transfer element 130. With the desired flow of each component gas calculated (block 311), the respective flow delivery valves are programmed to allow the calculated flow to pass (block 316).

It is determined whether the existing contents of differential flow transfer element 130 are to be modified or flushed as part of changing the gas mixture (block 326). Modifying the gas mixture includes adding component gases to the existing gas mixture in differential flow transfer element 130 until the desired mixture is achieved. In contrast, flushing differential flow transfer element 130 involves opening dump valve 140 to allow the current gas mixture in differential flow transfer element 130 to exhaust. Such a flushing process allows for a nearly immediate transformation from one gas mixture to the newly selected gas mixture. By modifying a gas mixture rather than flushing it, some savings can be achieved in component gases, however, the process introduces some delay in production of the newly requested gas mixture. In some cases, the determination of whether to modify or flush is based on a user input received via user interface 205. In one particular embodiment of the present invention, the default is to flush differential flow transfer element 130 unless an overriding user command is received along with the request for an updated gas mixture. In other embodiments of the present invention, determination of whether to modify or flush is based on calculating a time required to bring the gas mixture in differential flow transfer element 130 within the newly requested gas mixture request. If modification can be achieved within a prescribed time period, it may be automatically selected. The required time to modify the gas mixture may be calculated based on one or more of the present volume of the existing gas mixture in differential flow transfer element 130, the requested new gas mixture, the inlet rate(s) of a modification component gas, and the outlet rate from differential flow transfer element 130.

Thus, take for example a situation where the existing gas mixture is air with a twenty-two percent oxygen concentration by volume, differential flow transfer element 130 holds ‘n’ liters of the present gas mixture, the newly requested gas mixture is air with a twenty-three percent concentration of oxygen by volume, and no output of the gas mixture is currently occurring. In such a case, the time required to modify the existing gas mixture to yield the newly requested gas mixture is:

$$Time = \frac{[ \begin{matrix}( 1 - present oxygen concentration + \\ desired oxygen concentration ) n\end{matrix} ] - n}{oxygen flow rate} .$$

In this case where only the oxygen component gas is initially turned on, the calculated time may be small as the change in oxygen level is small and the oxygen flow rate may be reasonably high. The calculated time is the same where the inlet gas that is turned on includes both air component gas and oxygen component gas in relative flows to yield the twenty-three percent oxygen content by volume, although a greater volume of the combined gases is added to yield the desired mixture. In contrast, where the existing gas is heliox with ten percent oxygen, and the newly selected gas is air with a twenty-three percent oxygen content by volume, the calculated time will be relatively large and only achievable where some of the gas mixture is being produced from differential flow transfer element 130 to outlet 180. In such a case, a flush may be more reasonable. Based on the disclosure provided herein, one of ordinary skill in the art will recognize a variety of basis upon which a decision to flush or modify may be made.

Where a decision is made to flush differential flow transfer element 130 (block 326), dump valve 140 is opened and the existing gas mixture in differential flow transfer element 130 is exhausted (block 331). It is determined whether the flush is complete by, for example, reading pressure transducer 150 (block 336). Where it is not complete (block 336), dump valve 140 is maintained open. Alternatively, once the flush is complete (block 336), dump valve 140 is closed and the selected flow delivery valves 124 are turned on in proportion to the newly selected gas mixture to be produced by differential flow transfer element 130. Once this is complete, the interrupt process of flow diagram 301 is complete and control is returned to the inlet flow control process of flow diagram 300 as indicated by the ‘A’ designator.

Alternatively, where a decision is made to modify the gas mixture of differential flow transfer element 130 (block 326), one or more of flow delivery valves may be selectively turned on (block 346). Thus, following the above example of changing from a gas mixture of air with twenty-two percent oxygen by volume to a gas mixture of air with twenty-three percent oxygen by volume, only the oxygen component gas may be initially turned on. The oxygen component gas may be turned on at flow unrelated to that calculated in block 311 to yield a faster transformation, or at the flow calculated to yield the twenty-three percent oxygen volume when combined with air at a particular flow to yield a less complex transformation process. Alternatively, both the air component gas and the oxygen component gas may be turned on as programmed in block 316. Based on the disclosure provided herein, one of ordinary skill in the art will recognize a variety of inlet processes that may be used to modify the existing constituent concentrations of the gas in differential flow transfer element 130. Based on the chosen inlet flows and relative gas concentrations, it is determined whether sufficient time has passed to yield the desired gas mixture in differential flow transfer element 130 (block 351). Once it is determined that the mixture is as desired (block 351), the selected flow delivery valves 124 are turned on in proportion to the newly selected gas mixture to be produced by differential flow transfer element 130. Once this is complete, the interrupt process of flow diagram 301 is complete and control is returned to the inlet flow control process of flow diagram 300 as indicated by the ‘A’ designator.

Turning now to FIG. 3c, flow diagram 302 depicts a process producing the gas mixture from differential flow transfer element 130 to outlet 180. Following flow diagram 302, the ventilator incorporating differential flow transfer element 130 is turned on (block 307), and an outlet flow request is received (block 312). The outlet flow request may be entered by a user via user interface 205. The outlet flow request may be any ventilator outlet flow request known in the art. As one example, the outlet flow request may indicate a particular volume of the desired gas mixture to be delivered at a particular periodic interval. In some cases, the range of the exit flow request may extend from volume and rate parameters designed to satisfy the needs of a small neonatal patient up to those designed to meet the needs of a large adult male patient. Flow delivery valve 170 is programmed to meter the requested flow of the gas mixture from differential flow delivery valve 170 to outlet 180 (block 317), and flow delivery valve 170 is turned on to begin the flow (block 322). In some embodiments of the present invention, the gas flow through flow delivery valve 170 is maintained at a substantially constant rate that is much lower than the intermittent overall gas flow into differential flow transfer element 130. As such, the overall volume of gas inlet into differential flow transfer element 130 matches that outlet from differential flow transfer element 130 even though the inlet of gases involves relatively high flow rates over intermittent periods. The outlet flow continues until the ventilator is turned off (blocks 327, 332).

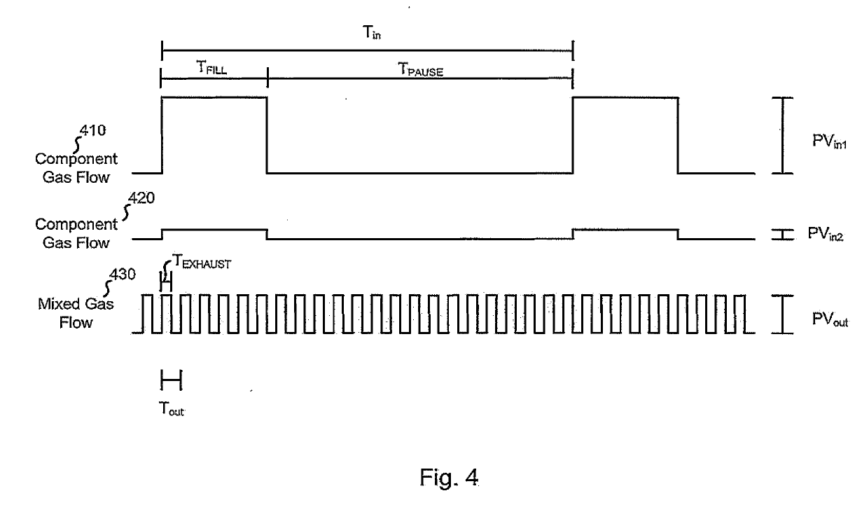

Turning to FIG. 4, a timing diagram 400 graphically depicts an example of an intermittent volume of component gas flow into a differential flow transfer element, and an intermittent volume of mixed gas flow from the differential flow transfer element that may be achieved in accordance with one or more embodiments of the present invention. As shown, two component gases represented as component gas flows 410, 420 are introduced into a differential flow transfer element, and a resulting mixed gas represented as a mixed gas flow 430 is outlet from the differential flow transfer element. The peak volume per unit time of component gas flow 410 is designated PVᵢₙ₁, and that of component gas flow 420 is designated as PVᵢₙ₂. The peak volume per unit time of mixed gas flow 430 is designated as PVₒᵤₜ. As shown, PVᵢₙ₁ is much greater than PVᵢₙ₂, and PVₒᵤₜ is less than PVᵢₙ₁ and greater than PVᵢₙ₂ per unit time. An inlet period (Tᵢₙ) consists of a fill period (T_{fill}) during which one or more component gases are flowing into the differential flow transfer element, and a pause period (Tₚₐᵤₛₑ) when the one or more gas flows are either zero or substantially reduced in comparison with that ongoing during T_{fill}. It should be noted, consistent with the discussion of FIGS. 3a-3c above, that Tᵢₙ, T_{fill} and Tₚₐᵤₛₑ may vary over time as gases are flowed into and out of differential flow transfer element. An outlet period is designated Tₒᵤₜ and includes an exhaust period, Tₑₓₕₐᵤₛₜ, when mixed gas flow 430 is flowing from the differential flow transfer element to an outlet. It should be noted that where one, three or more component gases are being incorporated into a mixed gas that more or fewer component gas flows would be represented.

It should be noted that as used herein, “flow rate” without more refers to the respective flows ongoing during T_{fill} and Tₑₓₕₐᵤₛₜ. Thus, the volume of gas provided to the differential flow transfer element for the inlet period would be:

Volume per Inlet Period=(PVᵢₙ₁+PVᵢₙ₂)T_{fill}=flow rate of component gas*T_{fill},

and the volume of gas provided from the differential flow transfer element for the outlet period would be:

Volume per Outlet Period=(PVₒᵤₜ)Tₑₓₕₐᵤₛₜ=flow rate of mixed gas*Tₑₓₕₐᵤₛₜ.

In contrast, when the phrase “average flow rate” is used, it is intended as described by the following equation:

$$Average Inlet Flow Rate = \frac{Volume per Inlet Period}{{T}_{i n}} ; and$$

$$Average Outlet Flow Rate = \frac{Volume per Outlet Period}{{T}_{out}} .$$

By increasing Tₚₐᵤₛₑ relative to T_{fill}, the overall peak inlet volume per unit time (i.e., PVᵢₙ₁+PVᵢₙ₂) can be substantially increased relative to the peak outlet volume per unit time (i.e., PVₒᵤₜ). This allows for increased component gas flows such that they fall within the accurately controlled range of given flow delivery valves. This accuracy is achieved without impacting the peak outlet volume per unit time that may be defined, for example, based on the particular needs of a recipient. It should be noted that the relative values of PVᵢₙ₁, PVᵢₙ₂, PVₒᵤₜ, Tᵢₙ, T_{fill}, Tₚₐᵤₛₑ, Tₒᵤₜ and Tₑₓₕₐᵤₛₜ are merely exemplary, and based on the disclosure provided herein, one of ordinary skill in the art will recognize a variety of relationships between the aforementioned parameters that may be programmed in accordance with different embodiments of the present invention. It should also be noted that where a dump valve is not opened to allow escape of mixed gases from within differential flow transfer element, that the overall inlet volume (i.e., [PVᵢₙ₁+PVᵢₙ₂]*t) will be approximately equal to the outlet volume (i.e., PVₒᵤₜ*t) where t is larger than the average inlet period. Additionally, it should be noted that while the periodicity of mixed gas flow 430 may be more regular than that of either component gas flow 410 or component gas flow 420, that it is not necessarily uniform due to, for example, the needs of a recipient that may vary over time. Such variance may be due to ventilator settings and recipient effort as is known in the art. It should also be noted that in some cases T_{fill} is not necessarily the same for each component gas, and does not necessarily occur concurrently for each component gas depending upon the particular application.

In conclusion, the invention provides novel systems, methods and devices for providing a defined gas flow to a recipient. While detailed descriptions of one or more embodiments of the invention have been given above, various alternatives, modifications, and equivalents will be apparent to those skilled in the art without varying from the spirit of the invention. Therefore, the above description should not be taken as limiting the scope of the invention, which is defined by the appended claims.

What is claimed is:

1. A gas delivery system, the gas delivery system comprising:
a differential flow transfer element, wherein the differential flow transfer element is coupled to a first component gas via a first flow valve and to a second component gas via a second flow valve, and wherein the differential flow transfer element is coupled to an outlet via a third flow valve;
a processor; and
a computer readable medium, wherein the computer readable medium includes instructions executable by the processor to:
operate the first flow valve intermittently at a first flow rate and the second flow valve intermittently at a second flow rate to yield a defined mixture including the first component gas and the second component gas in the differential flow transfer element; and
operate the third flow valve intermittently at a third flow rate to deliver the defined mixture including the first component gas and the second component gas from the differential flow transfer element to the outlet, wherein the third flow rate is less than the sum of the first flow rate and the second flow rate.

2. The gas delivery system of claim 1, wherein the differential flow transfer element is an accumulator.

3. The gas delivery system of claim 1, wherein over a period extending two or more inlet periods, the sum of the volume of the first component gas received via the first flow valve and the volume of the second component gas received via the second flow valve approximately equals the volume of the defined mixture provided via the third flow valve.

4. The gas delivery system of claim 1, wherein the computer readable medium further includes instructions executable by the processor to:
receive a request for the defined mixture; and
calculate the first flow rate and the second flow rate.

5. The gas delivery system of claim 4, wherein the defined mixture is a first defined mixture, and wherein the computer readable medium further includes instructions executable by the processor to:
receive a request for a second defined mixture including the first component gas and the second component gas; and
operate the first flow valve intermittently at a fourth flow rate and the second flow valve intermittently at a fifth flow rate to yield the second defined mixture of the first component gas and the second component gas in the differential flow transfer element.

6. The gas delivery system of claim 5, wherein the computer readable medium further includes instructions executable by the processor to:
open a dump valve to allow the contents of the differential flow transfer element to exhaust.

7. The gas delivery system of claim 1, wherein the computer readable medium further includes instructions executable by the processor to:
receive an indication of the pressure in the differential flow transfer element; and
calculate an amount of at least one constituent gas in the differential flow transfer element based at least in part on the pressure in the differential flow transfer element.

8. The gas delivery system of claim 1, wherein the computer readable medium further includes instructions executable by the processor to:
receive an indication of the volume of the first component gas traversing the first flow valve;
receive an indication of the volume of the second component gas traversing the second flow valve;
receive an indication of the volume of the defined mixture traversing the third flow valve; and
calculate an amount of at least one constituent gas in the differential flow transfer element based at least in part on the volume of the first component gas traversing the first flow valve, the volume of the second component gas traversing the second flow valve, and the volume of the defined mixture traversing the third flow valve.

9. A ventilator, the ventilator comprising:
a first gas source;
a second gas source;
a gas outlet; and
a differential flow transfer element that receives a first component gas from the first gas source at a first flow rate, receives a second component gas from the second gas source at a second flow rate, and provides a mixture including the first component gas and the second component gas at a third flow rate via the gas outlet; wherein the third flow rate is less than the sum of the first flow rate and the second flow rate.

10. The ventilator of claim 9, wherein over a period extending two or more consecutive inlet periods, the sum of the volume of the first component gas received from the first gas source and the volume of the second component gas received from the second gas source approximately equals the volume of the mixture provided via the gas outlet.

11. The ventilator of claim 9, wherein the first flow rate and the second flow rate are different.

12. The ventilator of claim 9, wherein the third flow rate exhibits a flow and periodicity consistent with a human breathing pattern.

13. The ventilator of claim 12, wherein at least one of the first flow rate and the second flow rate operates at a substantially higher flow than that of the third flow rate, but with a longer period than that of the third flow rate.

14. The ventilator of claim 9, wherein the differential flow transfer element is an accumulator operating at a pressure of between five and fifteen psi.

15. The ventilator of claim 9, wherein the differential flow transfer element receives the first component gas from the first gas source via a flow delivery module including a flow delivery valve, and wherein the flow delivery valve is programmable to deliver the first flow rate of the first component gas.

16. The ventilator of claim 15, wherein the flow delivery module further includes a flow sensor that is operable to sense the flow of the first component gas into the differential flow transfer element.

17. The ventilator of claim 9, wherein the differential flow transfer element provides the mixture of the first component gas and the second component gas to the outlet via a flow delivery valve.

18. The ventilator of claim 9, wherein the first component gas and the second component gas are selected from a group consisting of: air, oxygen, heliox, and helium.

19. A method for providing breathable gas to a recipient, the method comprising:
providing a ventilator with an accumulator, wherein the accumulator is coupled to a first component gas via a first flow valve and to a second component gas via a second flow valve, and wherein the accumulator is coupled to an outlet via a third flow valve;
receiving a request for a defined mixture including the first component gas and the second component gas;
operating the first flow valve intermittently at a first flow rate and the second flow valve intermittently at a second flow rate to yield the defined mixture in the accumulator; and
operating the third flow valve intermittently at a third flow rate to deliver the defined mixture from the accumulator to the outlet, wherein the third flow rate is less than the sum of the first flow rate and the second flow rate, and wherein over a period extending two or more inlet periods, the sum of the volume of the first component gas received via the first flow valve the volume of the second component gas received via the second flow valve approximately equals the volume of the defined mixture provided via the third flow valve.

20. The method of claim 19, the method further comprising:
receiving a request for the defined mixture; and
calculating the first flow rate and the second flow rate.

21. The method of claim 19, wherein the defined mixture is a first defined mixture, the method further comprising:
receiving a request for a second defined mixture including the first component gas and the second component gas; and
operating the first flow valve intermittently at a fourth flow rate and the second flow valve intermittently at a fifth flow rate to yield the second defined mixture in the accumulator element.

22. The method of claim 21, wherein the method further comprises:
opening a dump valve to allow the contents of the accumulator to exhaust.
